(19) United States
(12) Patent Application Publication
(10) Pub. No.: US 2026/0124650 A1
(43) Pub. Date: May 7, 2026

(54) Waste Treatment System

(71) Applicant: Cory Fletcher, Calgary (CA)

(72) Inventor: Cory Fletcher, Calgary (CA)

(21) Appl. No.: 18/934,365

(22) Filed: Nov. 1, 2024

(51) Int. Cl. B09B 3/35; A61L 2/10; A61L 2/26; A61L 11/00; B09B 3/32; B09B 3/50

(52) U.S. Cl. CPC B09B 3/35; A61L 2/10; A61L 2/26; A61L 11/00; B09B 3/32; B09B 3/50; A61L 2202/11

(57) ABSTRACT

A waste treatment system is provided. The waste treatment system includes an industrial shredder that processes non-recyclable waste into fine, sprayable material, an ultraviolet sterilizer that sanitizes the shredded waste material, and hydraulic rams that compress the material into solid construction blocks that secure to one another. The waste material is hydraulically compressed into a modular mold system that allows the production of blocks in various sizes. The blocks are reinforced with vertical bars and spacer rods, ensuring structural integrity. A male snap cap is secured to a first end of the bar and a female snap structure is secured to an opposing second end of the bar. The female snap structures are recessed at the bottom of each block to interlock securely with the male snap caps. The waste treatment system effectively reduces landfill waste and carbon footprint by converting waste into reusable construction materials.

### BACKGROUND OF THE INVENTION

The present invention relates to a waste treatment system designed to process non-recyclable waste into reusable construction materials. In the current technological landscape, managing non-recyclable waste is a significant challenge, with landfills and dumps accumulating vast amounts of waste that contribute to environmental degradation and carbon emissions. Existing waste management solutions, such as incineration and traditional landfilling, come with numerous drawbacks, including harmful emissions, inefficient use of space, and limited ability to repurpose the waste into useful products.

One existing method for dealing with waste involves shredding and compacting waste materials into bales for disposal or limited recycling applications. However, this approach often fails to produce materials that are versatile or structurally sound enough for construction purposes. The bales are usually bulky, prone to decomposition, and not suitable for long-term structural use. Moreover, the process of creating these bales does not adequately address the sterilization of waste, leading to potential health hazards and environmental contamination.

Another alternative involves the use of waste-to-energy plants, where non-recyclable waste is incinerated to produce energy. While this method reduces the volume of waste, it generates significant greenhouse gas emissions and toxic byproducts that require careful management. Additionally, waste-to-energy plants are expensive to build and operate, and they often face public opposition due to concerns about air pollution and health risks.

In the context of construction materials, current options such as concrete blocks, bricks, and prefabricated panels are primarily made from natural resources like sand, gravel, and clay. These materials require substantial energy and resources to produce, contributing to environmental degradation. Furthermore, the increasing demand for sustainable construction materials highlights the need for innovative solutions that can reduce reliance on virgin materials and minimize environmental impact. Therefore, there exists a need for a system configured to transform non-recyclable waste into high-quality construction blocks.

By converting waste into reusable construction materials, the waste treatment system significantly reduces the volume of waste sent to landfills, mitigates the environmental impact of traditional waste management practices, and provides a sustainable alternative to conventional building materials. This system not only addresses the pressing issue of waste management but also contributes to the development of sustainable construction practices, aligning with global efforts to reduce carbon footprints and promote environmental stewardship.

In light of the systems and methods disclosed in the known art, it is submitted that the present invention substantially diverges in design elements and methods from the known art and consequently it is clear that there is a need in the art for an improvement in waste treatment systems. In this regard the instant invention substantially fulfills these needs.

### SUMMARY OF THE INVENTION

In view of the foregoing disadvantages inherent in the known systems for waste treatment now present in the known art, the present invention provides a waste treatment system that converts waste into reusable construction materials.

It is an objective of the present invention to provide an embodiment of the waste treatment system that processes waste through a multistage industrial shredder that reduces the material to a fine, sprayable consistency. This fine material is then sterilized using ultraviolet light to ensure it is safe for handling and use. The sterilized waste is compacted into solid blocks using hydraulic rams capable of exerting pressure in excess of 10 gigatons, resulting in substantially voidless, blocks. Voidless herein refers to a state where the material, when pressed by a 10 gigaton hydraulic ram, is compacted to such a degree that it contains no air pockets, gaps, or internal cavities larger than 0.1 millimeters in diameter. This ensures uniform density and structural integrity throughout the entire block, enhancing its strength and durability for construction purposes.

It is another objective of the present invention to provide an embodiment of the waste treatment system comprising a modular mold setup that allows the production of blocks in various sizes, by changing a mold in the hydraulic press. Each block is reinforced with vertical round bars made of stainless steel, aluminum, or titanium, depending on the required load capacity. This reinforcement ensures the blocks are suitable for a wide range of construction applications, from temporary structures to permanent buildings.

It is another objective of the present invention to provide an embodiment of the waste treatment system comprising spray injection nozzles that coat each block with a protective layer of Duayen™, ensuring waterproofing and enhanced durability. This coating process distinguishes the waste treatment system from other waste management solutions by producing construction materials that are both environmentally friendly and structurally reliable.

It is an objective of the present invention to integrate precision torque motors into the waste treatment system for the installation of male snap caps on each block configured to secure to a female snap structure. This feature ensures that every block fits perfectly with an accuracy of 0.0001″, facilitating the construction of stable and precise structures.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings.

FIG. 1 shows a perspective view of an embodiment of the waste treatment system during manufacture.

FIG. 2 shows a perspective view of a block of an embodiment of the waste treatment system.

FIG. 3 shows a perspective view of a plurality of blocks of an embodiment of the waste treatment system.

### DETAILED DESCRIPTION OF THE INVENTION

Reference is made herein to the attached drawings. For the purpose of presenting a brief and clear description of the present invention, the preferred embodiment will be discussed as used for repurposing waste material into building construction material. The figures are intended for representative purposes only and should not be considered to be limiting in any respect.

Reference will now be made in detail to the exemplary embodiment(s) of the invention. References to “one embodiment,” “at least one embodiment,” “an embodiment,” “one example,” “an example,” “for example,” and so on indicate that the embodiment(s) or example(s) may include a feature, structure, characteristic, property, element, or limitation but that not every embodiment or example necessarily includes that feature, structure, characteristic, property, element, or limitation. Further, repeated use of the phrase “in an embodiment” does not necessarily refer to the same embodiment.

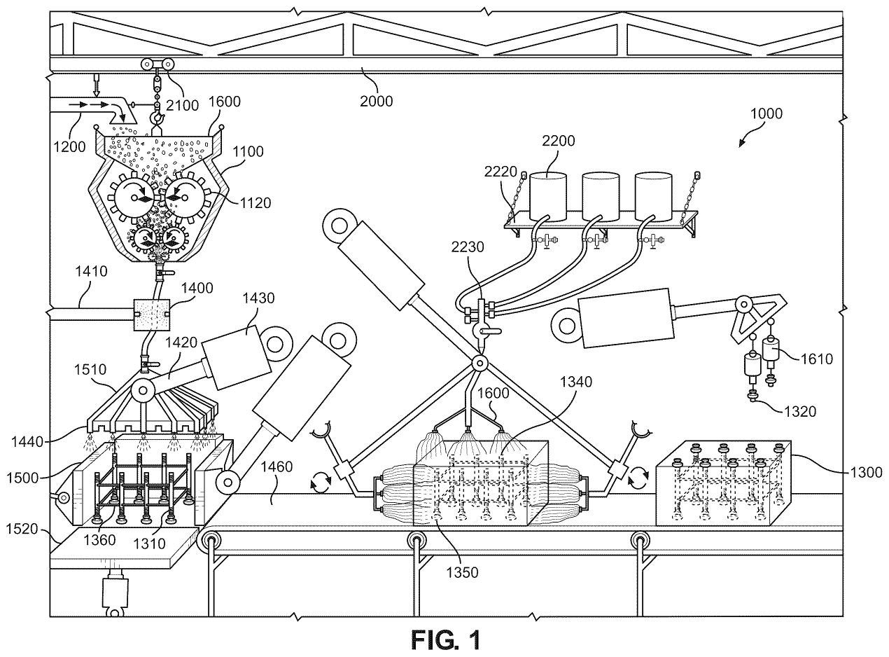

Referring now to FIG. 1, there is shown a perspective view of an embodiment of the waste treatment system during manufacture. The waste treatment system 1000 is designed for processing non-recyclable waste 6000 into reusable construction materials. The waste treatment system 1000 comprises a plurality of components for converting waste into solid, durable construction blocks 1300. The waste treatment system 1000 comprises an industrial shredder 1100 configured to process waste 6000 into a fine, sprayable consistency. Sprayable consistency is defined as the physical state of the shredded waste material, wherein the particle size is reduced to an average diameter of less than 2 millimeters. This consistency allows the material to be evenly dispersed and transported through a spray injection system without clogging or causing irregular flow patterns, ensuring uniform application during subsequent processing steps. In the illustrated embodiment, the industrial shredder 1100 is adapted to receive non-recyclable waste into a hopper that is received from a waste feeder 1200. The industrial shredder 1100 operates through multiple stages, each stage equipped with hardened steel blades 1120 capable of cutting through various materials, including plastics, textiles, and organic waste. The initial stage reduces the waste into larger fragments, while subsequent stages further reduce the size to achieve a consistency similar to smokable tobacco. The shredder 1100 is powered by high-torque electric motors, which ensure continuous and efficient operation. The shredding process includes an integrated dust and particulate control system to minimize airborne contaminants. In the illustrated embodiment, the industrial shredder 1100 is suspended from a crane structure 2000 via a chain 2100 to allow the system to be used on a construction site, wherein the blocks 1300 will be used as a building structure.

In some embodiments, an ultraviolet sterilizer 1400 is integrated to sanitize the processed waste material. Following the shredding process, the shredded waste material is conveyed to the ultraviolet (UV) sterilizer 1300. In the illustrated embodiment, the UV sterilizer 1300 uses high-intensity UV-C lamps, which emit wavelengths in the range of 200 to 280 nanometers, effectively destroying the DNA and RNA of microorganisms, ensuring 100% sterilization. The sterilizer 1300 comprises a conveyor system with an adjustable speed mechanism to control the exposure time of the material to UV light, ensuring thorough sterilization. The interior of the sterilizer is lined with reflective materials to enhance the efficacy of UV exposure, ensuring even and complete sterilization of the processed waste. The UV sterilizer 1300 is operably connected to a compressed air line 1410 to deliver an uninterrupted supply of compressed air to the system 1000. This step sanitizes the waste prior to pressurizing the line to make the waste into a sprayable form.

The sterilized waste material is then transferred to a hydraulic ram 1420, which compresses the material into solid blocks 1300, forming a voidless structure. The hydraulic ram 1420 is equipped with high-pressure cylinders 1430 capable of exerting forces exceeding 10 gigatons to achieve the desired density and structural integrity of each block 1300.

The waste treatment system 1000 comprises a modular mold system 1500 that allows the blocks 1300 to be produced in different sizes depending on the construction plan. In the illustrated embodiment, the modular mold system 1500 is adapted to produce blocks ranging in sizes from 6″×6″×6″ to 48″×96″×48″. The modular mold system 1500 comprises interchangeable molds made from high-strength steel alloys. Each mold comprises a compaction head 1510 and a plurality of mold walls 1520, each operably connected to a hydraulic ram 1420. These molds 1510, 1520 can be quickly swapped to produce blocks in various sizes. The molds are designed to withstand the high pressures involved in the compression process and feature a non-stick coating to facilitate the easy removal of the finished blocks. The sterilized waste material is sprayed via a plurality of spray nozzles 1440 into from the mold lid 1510 to fill the mold.

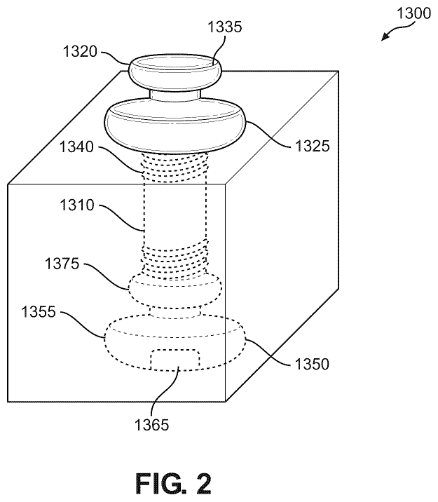

Referring now to FIGS. 1 and 2, there is shown the perspective view of an embodiment of the waste treatment system during manufacture and a perspective view of a block of an embodiment of the waste treatment system, respectively. Each block 1300 comprises at least one vertical bar 1310 integrated during the compression process to provide structural reinforcement. In the illustrated embodiment, the vertical bars are rounded and may be produced in various diameters and materials. In some embodiments, the bars 1310 are available in various diameters, ranging from ¼″ to 1″, and are made of materials such as stainless steel, aluminum, or titanium. The choice of material depends on the specific load requirements of the application. The bars 1310 are positioned within the molds before compression, ensuring they are perfectly aligned and evenly distributed throughout the block 1300. This reinforcement allows the blocks to support substantial loads, making them suitable for diverse construction applications, including load-bearing walls and structural supports. Each bar 1310 comprises a pair of threaded distal ends 1340 configured to receiving a male and a female snap structure 1320, 1350 that enable the blocks to secure to one another. In some embodiments, the waste treatment system comprises structural spacer rods 1360 maintain the vertical bars perfectly vertical and uniformly spaced within each block, ensuring consistent quality and strength. Each spacer 1360 extends perpendicularly between a pair of adjacent bars.

To connect the blocks to one another, male and female snap structures 1320, 1350 are disposed on opposing ends of the threaded bar 1310. In the illustrated embodiment, the male snap cap 1320 comprises a male cap member 1325 having an aperture disposed on a lower side thereof for threadedly engaging with the bar 1310. A mating member 1335 extends from an upper side of the male snap cap 1320 and is configured to insert within a mating recess 1365 of the female snap structure 1350. The mating member 1335 and the male cap member 1325 comprise a circular shaped cross section. The mating member 1335 comprises a smaller diameter than the diameter of the cap member 1325 of the male snap cap 1320.

Each block 1300 comprises the female snap structure 1350 at the bottom thereof, designed to interlock securely with the male snap caps 1320. In this way, a lowermost portion of the female snap structure 1350 is flush with the bottom of the block 1300. The female snap structure 1350 comprises a female cap member 1375 having an aperture disposed on an upper side thereof for threadedly engaging with the bar 1310. A female mating member 1355, having the mating recess 1365, extends from a lower side of the female snap structure 1320. The mating recess 1365 is disposed on a lower side of the female mating member 1355 and is configured to receive the mating member 1335 of the male snap cap 1320. The female mating member 1355 and the female cap member 1375 comprise a circular shaped cross section. The female mating member 1355 comprises a larger diameter than the diameter of the female cap member 1375 of the female snap structure 1350.

This interlocking mechanism provides a stable and secure connection between blocks, facilitating the construction of precise and robust structures. The design of the snap structures includes an audible snap sound to confirm correct and secure fastening, providing additional assurance during assembly. When installed, the bar 1310, and the male and female snap structures 1320, 1350 are aligned along a same vertical axis.

For precise assembly, the waste treatment system 1000 incorporates precision torque motors 1610 configured to install the male and female snap structures 1320, 1350 on each block 1300. These motors are equipped with high-resolution encoders and feedback systems, allowing for precise control of torque and positioning. The motors 1610 thread the snap caps 1320, 1350 onto the top or bottom of each block 1300 with an accuracy of 0.0001″, ensuring a perfect fit. This precision is essential for the stability and ease of assembly in construction applications, where the blocks interlock securely to form robust structures. The snap caps 1320 are made from durable materials such as stainless steel or high-strength polymers, ensuring long-term performance and reliability.

In the shown embodiment, a conveyor belt 1460 is used for efficient handling and processing of the waste material. Once the block is formed and released from the mold, it is transferred to the conveyor belt 1460. The blocks are then coated via a spray injection nozzle 1600 with a protective layer. In the illustrated embodiment, the protective layer is Duayen™ 2200. The spray injection nozzles 1600 apply a uniform coating of Duayen™ 2200 to each block 1300. Flow regulators 2230 and compressed air lines are stored on a shelf 2220, feeding the spray nozzles 1600. Duayen™ is a proprietary polymer-based coating known for its waterproofing and durability properties. The nozzles are designed to deliver a consistent flow of coating material, ensuring even coverage of the block's surface. The coating process involves multiple passes to build up a sufficient layer thickness, enhancing the block's resistance to environmental factors such as moisture, UV radiation, and abrasion. The spray system includes automated controls to monitor and adjust the flow rate and coating thickness in real-time.

In the illustrated embodiment, the waste treatment system 1000 is powered by an electromagnetic generator, providing a sustainable and independent power source for all processing stages. The electromagnetic generator operates on the principles of electromagnetic induction, converting mechanical energy into electrical energy. This feature ensures that the system can operate efficiently in various locations, including remote areas without access to conventional power sources. The generator is designed to provide a stable and continuous power supply, capable of meeting the high energy demands of the shredding, sterilizing, and compression processes.

The waste treatment system supports both portable and permanent construction applications. For portable structures, the blocks are assembled with a single Duayen™ coating. Permanent structures receive an additional Duayen™ coating on each side of the constructed wall, increasing stability and guaranteeing waterproofing. The transition between configurations involves applying the additional coating to the blocks once the structure is assembled. This process enhances the durability and resistance of the structure to environmental factors, making it suitable for long-term use in various climates and conditions.

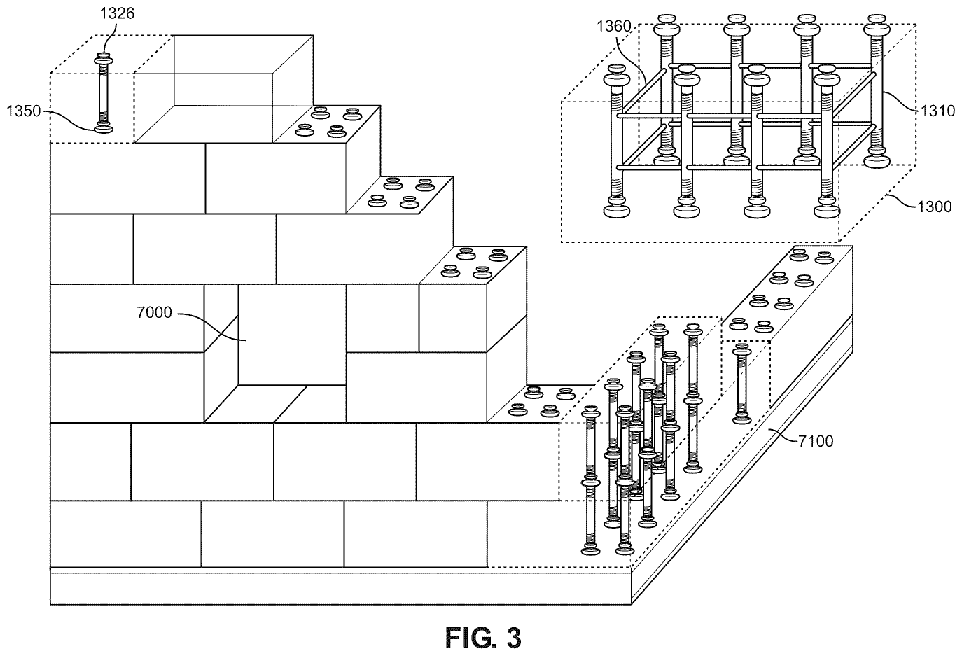

Referring now to FIG. 3, there is shown a perspective view of a plurality of blocks of an embodiment of the waste treatment system. A method for processing non-recyclable waste into reusable construction materials using the waste treatment system 1000 comprises first collecting and sorting non-recyclable waste to remove recyclable and hazardous materials. The waste material is then fed into an industrial shredder to reduce the waste into a fine, sprayable consistency through multiple stages of shredding. The shredded waste material is then gravity fed to an ultraviolet sterilizer and exposed to high-intensity UV-C light to achieve 100% sterilization. The sterilized waste material is then transferred to hydraulic rams, wherein the material is compressed into solid construction blocks using high-pressure cylinders and a modular mold setup. The vertical bars are integrated into the construction blocks during the compression process, before the sterilized waste material is sprayed into the mold. The compressed construction blocks are then sprayed with a uniform layer of Duayen™ using spray injection nozzles to ensure waterproofing and durability, wherein the coated blocks are transported along a conveyor belt for final processing and quality checks. Both male and female snap structures are installed onto each block using precision torque motors to achieve a fit accuracy of 0.0001″, ensuring the blocks include recessed female snap structures at the bottom to interlock securely with the male snap caps. In some embodiments, the block receives an additional layer of Duayen™ coating for permanent structures to enhance stability and waterproofing.

In the illustrated embodiment, during installation of a building structure using the waste treatment system 1000, a floor 7100 forming the base of the structure must be level to achieve a level structure using the blocks 1300. Windows, doors, and other openings 7000 can be formed within the building structure, as well.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A waste treatment system, comprising:
a shredder configured to process waste into a sprayable consistency, defined as having an average particle diameter of less than 2 millimeters;
an ultraviolet sterilizer configured to receive and sanitize the processed waste;
a modular mold system having a mold compaction head and a plurality of walls, wherein a hydraulic ram is connected to the mold compaction head and the plurality of walls configured to compress the sterilized waste into a block;
a bar having a first end and an opposing second end, wherein the bar is integrated into the block;
a female snap structure disposed on the first end of the bar and recessed within a lower side of the block;
a male snap cap disposed on the second end of the bar, wherein the male snap cap is exterior to an upper side of the block;
wherein the female snap structure of the block is configured to receive a male snap cap of a second block and the male snap cap of the block is configured to insert into a female snap structure of a third block.

2) The waste treatment system of claim 1, wherein the industrial shredder includes multiple stages to progressively reduce the particle size of the waste to the sprayable consistency, wherein each stage comprises a different sized blade.

3) The waste treatment system of claim 1, wherein the ultraviolet sterilizer is configured to achieve sterilization of the waste material prior to compaction.

4) The waste treatment system of claim 1, wherein the hydraulic ram exerts a pressure in excess of 10 gigatons configured to prevent an internal cavity larger than 0.1 millimeters in diameter.

5) The waste treatment system of claim 1, further comprising a spray injection nozzle for coating the block with a protective layer configured to waterproof the block.

6) The waste treatment system of claim 5, wherein the protective layer is composed of a uniform coat of Duayen™.

7) The waste treatment system of claim 1, wherein mold compaction head and the plurality of walls are independent from one another, allowing each of the plurality of walls to be interchanged with a different sized wall.

8) The waste treatment system of claim 7, wherein the modular mold system is configured to produce a block in various sizes ranging from 6″×6″×6″ to 48″×96″×48″.

9) The waste treatment system of claim 1, wherein the waste treatment system is powered by an electromagnetic generator.

10) The waste treatment system of claim 1, further comprising a spacer rod disposed perpendicularly between a pair of adjacent bars.

11) The waste treatment system of claim 1, further comprising a precision torque motor for the installation of the male snap cap on the block.

12) The waste treatment system of claim 11, wherein the precision torque motor is configured to thread the male snap cap onto each block.

13) The waste treatment system of claim 1, wherein the male snap cap comprises a male cap member and a mating member, the male cap member having an aperture disposed on a lower side thereof for threadedly engaging with the bar and the mating member extending from an upper side of the male cap member configured to insert within a mating recess of the female snap structure.

14) The waste treatment system of claim 13, wherein the mating member comprises a smaller diameter than a diameter of the male cap member.

15) The waste treatment system of claim 13, wherein the female snap structure comprises a female cap member and a female mating member, the female cap member having an aperture disposed on an upper side thereof for threadedly engaging with the bar and the female mating member having the mating recess.

16) The waste treatment system of claim 15, wherein the female mating member extends from a lower side of the female cap structure and the mating recess is disposed on a lower side of the female mating member.

17) The waste treatment system of claim 16, a lowermost portion of the female snap structure is flush with the bottom of the block.

18) A block produced by the process comprising:
processing waste into a sprayable consistency, defined as having an average particle diameter of less than 2 millimeters, using a shredder;
sanitizing the processed waste using an ultraviolet sterilizer;
compressing the sterilized waste into a block using a modular mold system, wherein the mold system includes a mold compaction head and a plurality of walls, and a hydraulic ram connected to the mold compaction head;
integrating a bar into the block, wherein the bar has a first end and an opposing second end;
disposing a female snap structure on the first end of the bar and recessing it within a lower side of the block;
disposing a male snap cap on the second end of the bar, wherein the male snap cap is exterior to an upper side of the block;
wherein the female snap structure of the block is configured to receive a male snap cap of a second block, and the male snap cap of the block is configured to insert into a female snap structure of a third block.

19) The block of claim 18, wherein the mold compaction head and the plurality of walls are independent from one another, allowing each of the plurality of walls to be interchanged with different sized walls.

20) The block of claim 1, further comprising a spacer rod disposed perpendicularly between a pair of adjacent bars.
